(19) United States
(12) Patent Application Publication
(10) Pub. No.: US 2023/0360748 A1
(43) Pub. Date: Nov. 9, 2023

(54) ELECTRONIC MEDICAL RECORDS AND TEMPLATE SYSTEM

(71) Applicant: SKINTAP INC., GREAT FALLS, VA (US)

(72) Inventors: BRANDON VAUGHAN, Ashburn, VA (US); WILLIAM GIANDONI, FALLS CHURCH, VA (US)

(21) Appl. No.: 17/737,223

(22) Filed: May 5, 2022

(51) Int. Cl. G16H 10/60

(52) U.S. Cl. CPC G16H 10/60

(57) ABSTRACT

Various embodiments for customizing electronic medical records system are described herein. An embodiment operates by receiving a request from a doctor to generate a medical diagnosis for a patient. One or more preconfigured templates corresponding to the medical diagnosis input by the doctor are identified. A selection of at least one of the identified one or more preconfigured templates is received. Text of the selected at least one of the identified one or more preconfigured templates is copied into a treatment plan for the patient. The treatment plan including at least a portion of the copied text of the selected at least one of the identified one or more preconfigured templates is provided to the patient.

### BACKGROUND

With insurance companies putting increasing price pressures on doctors, doctors often have less time to spend on each patient if they want to remain profitable and in business. Also, the less time a doctor spends on any particular patient, the more patients the doctor can see. However, most patients will still require a basic level medical care that will include an exam and a medical diagnosis. It would be beneficial for doctors, patients, and insurance companies to have a system that enables doctors to more quickly provide high quality care to patients, particularly in performing the medical diagnosis.

### BRIEF DESCRIPTION OF THE FIGURES

The accompanying drawings are incorporated herein and form a part of the specification.

FIG. 1 illustrates a block diagram of an electronic medical records system (EMRS), according to some example embodiments.

FIG. 2 illustrates a block diagram illustrating example templates of an electronic medical records system (EMRS), according to some example embodiments.

FIG. 3 is a flowchart illustrating a process for an electronic medical records system (EMRS), according to some embodiments.

FIG. 4 illustrates a block diagram of a provisioning and debugging system, according to some additional example embodiments.

In the drawings, like reference numbers generally indicate identical or similar elements. Additionally, generally, the left-most digit(s) of a reference number identifies the drawing in which the reference number first appears.

### DETAILED DESCRIPTION

With insurance companies putting increasing price pressures on doctors, doctors often have less time to spend on each patient if they want to remain profitable and in business. Also, the less time a doctor spends on any particular patient, the more patients the doctor can see. However, most patients will still require a basic level medical care that will include an exam and a medical diagnosis. It would be beneficial for doctors, patients, and insurance companies to have a system that enables doctors to more quickly provide high quality care to patients, particularly in performing the medical diagnosis.

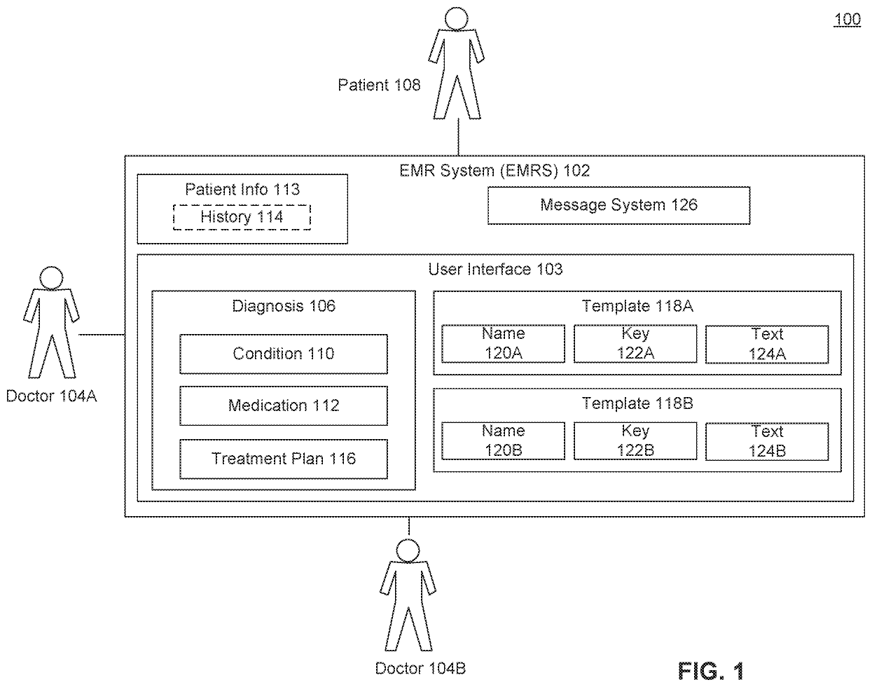

FIG. 1 illustrates a block diagram 100 of an electronic medical records system (EMRS) 102, according to some example embodiments. EMRS 102 may provide a user interface 103 and features that simplify how a doctor 104 completes a medical diagnosis 106 after a medical exam and communicates with a patient 108. EMRS 102 may provide a streamlined computing system and user interface 103 that enables a doctor 104 to quickly complete a medical diagnosis 106. EMRS 102 may also provide various templates 118 that a doctor 104 can use to simplify or streamline communications with the patient 108.

In some embodiments, EMRS 102 may allow a patient 108 to upload their patient info 113 into EMRS 102. Patient info 113 may include any information about the patient and/or the current problem or condition for which the patient 108 is seeking medical care. Example patient info 113 includes, but not limited to, name, insurance, age/date of birth, sex, what symptoms they are experiencing, images, notes, address, medications, other illnesses, and other information. Patient 108 may use a mobile phone or laptop to access a patient interface of EMRS 102 over the Internet. The patient interface may prompt the patient 108 for various information some of which may be required or optional, which is then stored as patient info 113. In some embodiments, patient info 113 may be imported from a database or other electronic records system, as well as from patient 108.

EMRS 102 may make patient info 113 accessible to doctor 104A who may then perform a medical exam with patient 108. The medical exam may be an in-person doctor visit or a video/telephone call. In some embodiments, the medical exam may be performed by the doctor asynchronously based on the uploaded patient info 113. For example, doctor 104A may be a dermatologist (or other specialist) and the patient may upload pictures, videos, and/or descriptions of the symptoms the patient has been experiencing, length of time, medications used, etc. And based on the uploaded information, the doctor may perform their medical exam and determine a medical diagnosis (e.g., virtual visit).

In some embodiments, EMRS 102 may provide a messaging system 126 that enables doctor 104A to communicate directly with patient 108 and request additional information beyond what was provided in patient info 113 and/or was received during medical exam. Messaging system 126 may be a secured messaging system between doctor 104A and patient 108 through EMRS 102 in which they may exchange medical information. Messaging system 126 may enable for text, photos, videos, or even voice messages to be exchanged between doctor 104A and patient 108. In some embodiments, messages from messaging system 126 may be stored as part of history 114, which may be a medical history of the patient 108. In other embodiments, the messages may automatically be deleted after a set period of time.

EMRS 102 may provide doctor 104A with user interface 103 that enables the doctor 104A to quickly complete a diagnosis 106 as part of or after the medical exam with patient 108 (or based on patient info 113). Diagnosis 106 may be an electronic version of the medical diagnosis of the sickness, illness, injury, disease, cause, cosmetic issue, and/or treatment of patient 108.

Condition 110 may indicate what medical condition(s) that the doctor 108A has diagnosed for patient 108 based on the medical exam. The medical condition may include any disease, injury, sickness, illness, or cosmetic recommendation. User interface 103 may list or sort various selectable conditions 110 in an order that is going to be most beneficial for doctor 104A, e.g., that is going to enable doctor to most quickly be able to identify and select one or more conditions 110 that pertain to patient 108.

In some embodiments, EMRS 102 may configure user interface 103 based on a specialty of the doctor 104 performing the diagnosis 106 (doctor 104 is a term used generally to refer to doctor 104A and/or doctor 104B). For example, if doctor 104A is a dermatologist, then EMRS 102 may filter out only those conditions 110 that are most relevant to dermatologists, thus decluttering and simplifying the user interface 103 by automatically providing the doctor 104A with fewer conditions 110 from which to choose. The doctor 104A may still be able to search for other non-dermatological conditions, but they may not be listed on user interface 103 without the doctor 104 first performing a specific search for those non-dermatological conditions. It is understood that while dermatology is used as an example, doctor 104A may be any type of doctor in any medical specialty, and EMRS 102 may be configured for that particular specialty.

In some embodiments, EMRS 102 may automatically assign requests received from new patients 108 to a doctor 104 based on the availability or patient load of the doctor 104. For example, a medical practice may have multiple doctors 104 with the same specialty (e.g., dermatology). EMRS 102 may receive a request from a new patient for a dermatology visit, and may automatically select one of the doctors 104 based on their immediacy of availability or patient workload (i.e., the doctor with the soonest availability or lightest patient workload may be selected).

Existing or returning patients may be assigned to their previous doctors 104. In some embodiments, returning patients may be provided an option to be examined by a different doctor 104 if their previous doctor 104 is unavailable for a threshold period of time (e.g., 2 weeks). For example, if a doctor 104A is busy, out of town, on vacation, or a leave of absence, EMRS 102 may respond to new requests for that doctor 104A to the patient with an indication (e.g., display notification) that the doctor is unavailable, and the patient may be prompted to choose from a list of available doctors 104 or authorize EMRS 102 to select a new doctor 104 for the visit.

In some embodiments, EMRS 102 may have access to a patient history 114. In some embodiments, patient history 114 may be a medical history for a particular patient 108 or a history across various patients whom the doctor 104A (or a medical practice with which the doctor is employed) has treated. EMRS 102 may then sort, order, or arrange the conditions 110 based on which conditions 110 are found in the patient history 114. For example, EMRS 102 may sort the most frequently and/or recently diagnosed conditions 110 near the top or with a variance in visual characteristics. For example, previous conditions 110 for the particular patient 108 may appear near the top or with varying visual characteristics relative to other non-previously diagnosed or selected conditions 110 for patient 108.

Examples of changes in visual characteristics may include indicating a previously diagnosed medical condition 110 with a different color (red) and/or indicate a date when the previous diagnosis was made, or increasing the size of text of a frequently selected medical condition. In some embodiments, history 114 may include diagnosis of patient 108 made by other doctors 104. In these situations, the previously diagnosed conditions 110 may indicate the previous doctor name or clinic. Or for example, conditions diagnosed by a different doctor or clinic may appear in a different color than conditions diagnosed by the presently treating doctor 104.

After the doctor 104A selects one or more conditions 110, EMRS 102 may streamline or declutter user interface 103 to make selecting one or more medications faster or easier for doctor 104A as well. For example, EMRS 102 may automatically (with or without a request from doctor 104A) filter or sort a list of medications 112 that are frequently prescribed for with the selected condition(s) 110 or the type of medical practice (e.g., dermatology). This determination of which medications are likely to be prescribed may be based on tracking data across various doctors 104, from a medical database (not shown), or from a tracking of the specific doctor's own prescription history 114 for the selected condition(s) 110. These most likely to be prescribed medications may be filtered or sorted to the top of the list of medications, bolded, or increased in size, or include another changed visual characteristic making it faster for the doctor to see and select from user interface 103.

Additionally, or alternatively, EMRS 102 may enable doctor 104A to scroll the various selectable medications 112 in alphabetical order or type in the name for an automatic search using a search bar. In some embodiments, EMRS 102 may determine based on history 114 which medications 112 have been previously prescribed to the patient, when, and by which doctor and/or for which conditions. EMRS 102 may then provide visual indicators of these medications on user interface 103, this may include sorting these previous medications to the top of a list of medications and indicating a most recent date of prescription.

Oftentimes simply telling a patient what condition or illness they have been diagnosed with and prescribing a set of one or more medicines does not provide a high enough level of care for the patient 108. It may often be the case that the patient needs more information about the condition and/or medications they are taking for proper care and treatment. EMRS 102 enables doctor 104A to provide any additional information specific to patient regarding their patient info 113, history 114, condition 110, or medication 112 through the form of a treatment plan 116.

A treatment plan 116 may include the selected conditions 110, selected medications 112, and any additional doctor provided input or feedback to the patient 108, which may include any free text typed or spoken by doctor 104A. Without EMRS 102, a doctor would individually write up notations for the patient every single time there was a condition or medication. And if multiple patients were each diagnosed with the same conditions or prescriptions (medications), the doctor would write or type essentially the same content or free text over and over and over again as part of a treatment plan or note for each patient.

To simplify and speed up the completion of diagnosis 116, particularly treatment plan 116, EMRS 102 may provide various templates 118 that enable the doctor 104A to re-use notations to patients across different patients. Template 118 is used generally to refer to one or both of template 118A and template 118B. Template 118 may be a previously used or prewritten notation for a patient that pertains to a specific condition 110, medication 112, or portion of patient info 113 (or patient history 114), such as age, sex, location, pre-existing conditions, etc.

In some embodiments, a template 118 may include a name 120, key 122, and text 124. Name 120 (used to generally refer to name 120A, 120B) and may be a user or doctor provided name of the template that communicates to a doctor 104 when to use or select the template 118B. The name 120 may be used to alphabetically sort the templates 118.

Text 124 may include the text and/or images of the template 118 that are copied into or otherwise included as part of the treatment plan 116 when the template 118 is selected. In some embodiments, text 124 may include doctor-provided instructions or warnings about the selected medication(s) 112 or how to treat the selected condition(s) 110. For example, if a particular medication 112 has drowsiness as a side effect, the text 124 may instruct the patient not to take while driving or to take it close to bedtime. Text 124 is used generally to refer to text 124A and/or text 124B.

Key 122 may be any conditions 110, medications 112, patient info 113, information from history 114 that may be used to indicate that the particular template is relevant and likely to be selected by doctor 104A. EMRS 102 may use key 122 (which refers generally to key 122A and/or key 122B) to determine when to highlight (e.g., bold, change color, increase size) or sort near the top of a list of selectable templates a particular template 118.

For example, key 122A for template 118A may include the condition rosacea, while the keys 122B for template 118B may include the terms ibuprofen and headache. Then for example, if EMRS 102 detects that rosacea is selected as a condition 110, the template 118A may be provided near or at the top of the list of selectable templates (even if is name is alphabetically after template 118B). In some embodiments, EMRS 102 may automatically copy text 124A into treatment plan 116 for any detected key 122.

If there are multiple templates 118 that all indicate the same key 118, such as rosacea, then all those templates 118 may be provided near the top of the list or highlighted or bolded. Or, for example, if key 122A includes condition A and medication B, and key 122B includes condition A and medication C, and EMRS 102 detects a selection of condition A as condition 110, and medication C as medication 112, template 118B may be provided with an indication of a greater priority than template 118A (which still may be provided with a greater priority than other less relevant templates 118 with no matching keys 122 to the selected conditions 110 and/or medications 112, or that do not match patient info 113). As indicated above, the indicator of a greater priority may be a sorting the template 118 to the top of a list, and/or changing the visual appearance (e.g., text size, color, font) of a name 120 of a template 118.

This high priority indicator of templates 1189 may enable a doctor 104A to more quickly see and select which template(s) 118 to use in treatment plan 116, which both increases the usability of user interface 103 (e.g., by filtering out unnecessary or potentially irrelevant information, thus decluttering the user interface 103) and shortens the time required for diagnosis 106. The doctor 104A may then update or edit the copied text 124 in treatment plan 116 for the particular patient 108, if needed. In some embodiments, EMRS 102 may enable doctor 104A to select multiple templates 118 and decide the order in which they are included in treatment plan 116 using drag-and-drop commands prior to or after they are copied into treatment plan 116.

Doctor 104A may then optionally edit/customize the copied template(s) 108 in treatment plan 116 before finalizing or submitting the treatment plan. In some embodiments, different conditions 110 and/or medications 112 may include different treatment plans 116 for the same patient 108 as part of the same medical visit or exam.

In some embodiments, doctor 104A may select one or more templates 118 to use in writing messages to patient 108 through message system 126. For example, if a patient asks a frequently asked question, there may be a relevant template 118 that the doctor 104A can select, optionally edit/customize, and send to patient 108.

In some embodiments, EMRS 102 may enable different doctors 104A, 104B in the same medical practice, or across different medical practices to create and share templates 118. For example, doctor 104B may create template 118A and share it with doctor 104A.

Doctor 104A may then use template 118A as is, or edit the template 118A for his own personal use. In some embodiments, doctor 104A may edit the template 118A and share those edits with doctor 104B (and any other doctors 104 who are using the template 118A, or other doctors 104 not yet using template 118A). EMRS 102 may indicate which portion(s) of template 118A were changed (e.g., name 120A, key 122A, and/or text 124A) by changing the color of those portions and/or indicating with underlines for added text and strikethroughs for deleted text. Each doctor 104 may then have the option of accepting the changes by doctor 104A, rejecting the changes, editing the changes, and/or saving the changes to a new version of template 118A for their personal use.

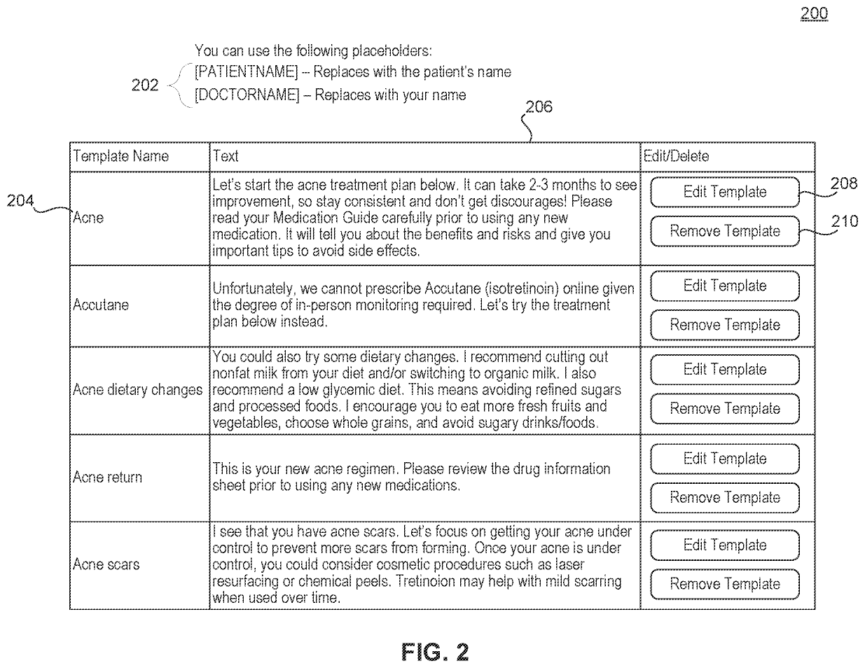

FIG. 2 illustrates a block diagram 200 illustrating example templates of an electronic medical records system (EMRS) 102, according to some example embodiments. FIG. 2 illustrates five example templates (e.g., templates 118) that a doctor 104 can either edit 208 or remove 210. Each template has a name 204 and body text 206.

The illustrated templates are displayed in alphabetical order by name 204, however as noted above, in a real-time system, when a doctor 104 is filling out a diagnosis 106, the sort or order of the templates 118 may be changed based on which conditions 110 and/or medications 112 are selected.

In some embodiments, the block diagram 200 may also include key information 122 that is used to perform the real-time sort. If no key information is provided for templates 118, those templates may be provided in alphabetical order based on the template name 204.

As illustrated in 202, in some embodiments, a template play include placeholder information that is filled in during real-time by EMRS 102 when the template is selected. For example, if a particular template includes the [PATIENTNAME] placeholder, then when the template is selected, [PATIENTNAME] may be replaced with the first name of the patient as derived from patient info 113.

In some embodiments, doctor 104 may customize what name is filled in for the placeholder [DOCTORNAME]. For example, if a doctor's name is Edward Jones. The doctor may customize their name as “Doctor Ed Jones”, “Dr. Jones”, or “Dr. Edward Jones”, as a few examples, which may be stored by EMRS 102.

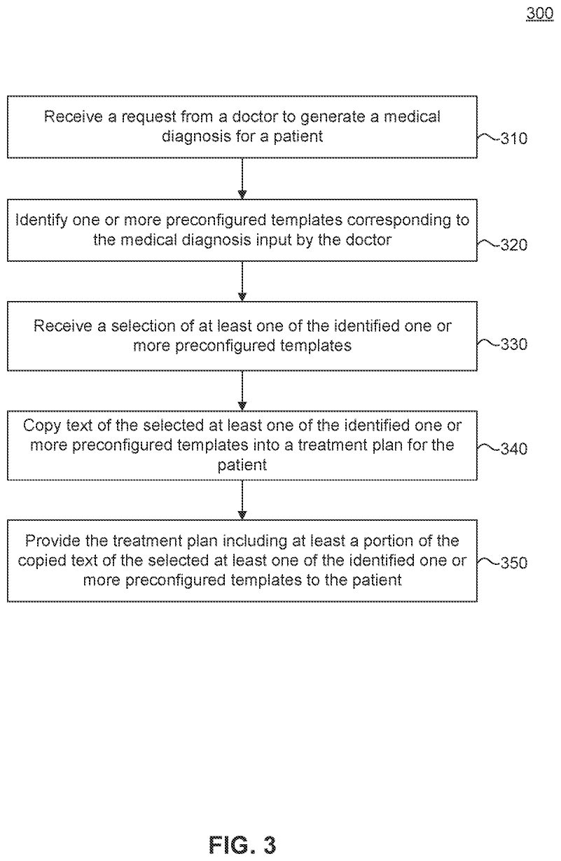

FIG. 3 is a flowchart illustrating a process 300 for a an electronic medical records system (EMRS) 102, according to some embodiments. Method 300 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 3, as will be understood by a person of ordinary skill in the art. Method 300 shall be described with reference to FIG. 1. However, method 300 is not limited to that example embodiment.

In 310, a request is received from a doctor to generate a medical diagnosis for a patient. For example, EMRS 102 may receive a selection of a button to generate diagnosis 106 in user interface 103. The diagnosis 106 may be performed during, as part of, or after a medical exam with patient 108. The medical exam may have been in person, through video, or based on electronically submitted or retrieved patient info 113, which may include all the information necessary to perform diagnoses (e.g., such as answers to various questions about the symptoms, free text by patient 108, and/or images). If doctor 104A requires additional information, the doctor 104A may send a message to patient 108 using messaging system 126.

In 320, one or more preconfigured templates corresponding to the medical diagnosis input by the doctor are identified. For example, doctor 104A may select a condition 110 and/or a medication 112, and EMRS 102 may filter out the most likely relevant templates 118 based on the doctor's selections. In some embodiments, the relevancy determination may be based on matching keys 122A, 122B with the selection of condition 110 and medication 112 in diagnosis 106. As used herein, the term doctor 104 may include any personnel who work with or for doctor 104 who are authorized to provide medical information to EMRS 102.

In 330, a selection of at least one of the identified one or more preconfigured templates is received from the doctor. For example, EMRS 102 may provide a list of various filtered, sorted, and selectable templates 118 for the doctor 104A to choose from. The doctor 104A may then use check boxes or drag-and-drop commands to select one or more of the templates 118 that are relevant to patient 108.

In 340, text of the selected at least one of the identified one or more preconfigured templates is copied into a treatment plan for the patient. For example, EMRS 102 may copy the text 124 of any selected template 118 into treatment plan 116. Doctor 104A may then edit the text 124 to customize it for the patient 108, if needed.

In 350, the treatment plan including at least a portion of the copied text of the selected at least one of the identified one or more preconfigured templates to the patient is provided to the patient. For example, doctor 104A may hit a submit button in user interface 103. Upon receiving the selection of the submit button, EMRS 102 may provide the treatment plan 116 as a message through messaging system 126. In some embodiments, EMRS 102 may provide treatment plan 116 via email, text message, or a selectable hyperlink. In some embodiments, EMRS 102 may automatically and electronically transmit the selected medications 112 to a pharmacy for fulfillment for patient 108 upon receiving the selection of the submit button.

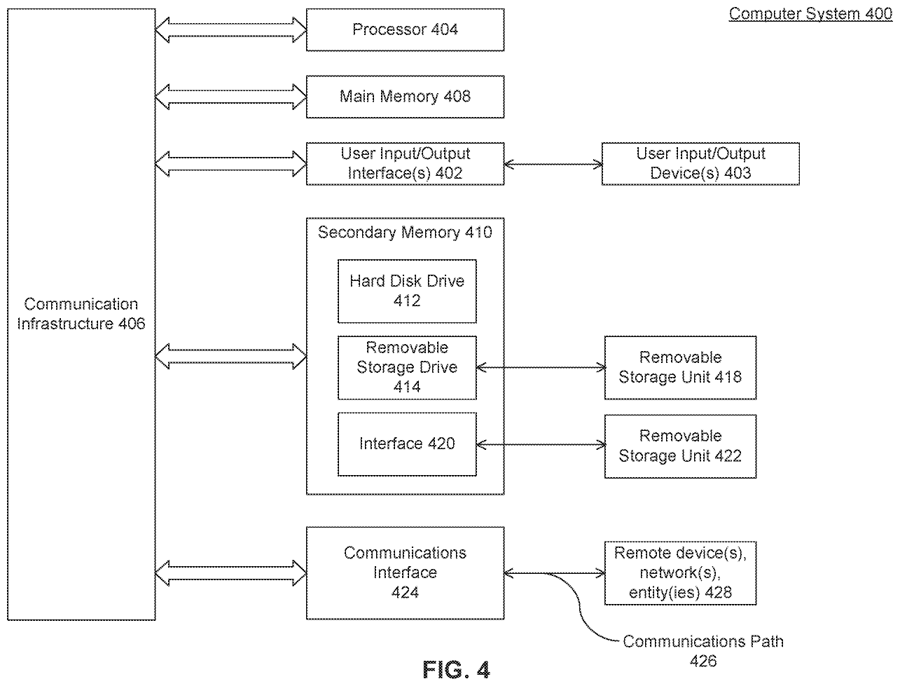

Various embodiments and/or components therein can be implemented, for example, using one or more computer systems, such as computer system 400 shown in FIG. 4. Computer system 400 can be any computer or computing device capable of performing the functions described herein. For example, one or more computer systems 400 can be used to implement any embodiments of FIGS. 1-3, and/or any combination or sub-combination thereof.

Computer system 400 includes one or more processors (also called central processing units, or CPUs), such as a processor 404. Processor 404 is connected to a communication infrastructure or bus 406. Computer system 400 may represent or comprise one or more systems on chip (SOC).

One or more processors 404 can each be a graphics processing unit (GPU). In some embodiments, a GPU is a processor that is a specialized electronic circuit designed to process mathematically intensive applications. The GPU can have a parallel structure that is efficient for parallel processing of large blocks of data, such as mathematically intensive data common to computer graphics applications, images, videos, etc.

Computer system 400 also includes user input/output device(s) 403, such as monitors, keyboards, pointing devices, etc., that communicate with communication infrastructure 406 through user input/output interface(s) 402.

Computer system 400 also includes a main or primary memory 408, such as random access memory (RAM). Main memory 408 can include one or more levels of cache. Main memory 408 has stored therein control logic (i.e., computer software) and/or data.

Computer system 400 can also include one or more secondary storage devices or memory 410. Secondary memory 410 can include, for example, a hard disk drive 412 and/or a removable storage device or drive 414. Removable storage drive 414 can be a floppy disk drive, a magnetic tape drive, a compact disk drive, an optical storage device, tape backup device, and/or any other storage device/drive.

Removable storage drive 414 can interact with a removable storage unit 418. Removable storage unit 418 includes a computer usable or readable storage device having stored thereon computer software (control logic) and/or data. Removable storage unit 418 can be a floppy disk, magnetic tape, compact disk, DVD, optical storage disk, memory card, and/any other computer data storage device. Removable storage drive 414 reads from and/or writes to removable storage unit 418 in a well-known manner.

According to an exemplary embodiment, secondary memory 410 can include other means, instrumentalities or other approaches for allowing computer programs and/or other instructions and/or data to be accessed by computer system 400. Such means, instrumentalities or other approaches can include, for example, a removable storage unit 422 and an interface 420. Examples of the removable storage unit 422 and the interface 420 can include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 400 can further include a communication or network interface 424. Communication interface 424 enables computer system 400 to communicate and interact with any combination of remote devices, remote networks, remote entities, etc. (individually and collectively referenced by reference number 428). For example, communication interface 424 can allow computer system 400 to communicate with remote devices 428 over communications path 426, which can be wired and/or wireless, and which can include any combination of LANs, WANs, the Internet, etc. Control logic and/or data can be transmitted to and from computer system 400 via communication path 426.

In some embodiments, a tangible apparatus or article of manufacture comprising a tangible computer useable or readable medium having control logic (software) stored thereon is also referred to herein as a computer program product or program storage device. This includes, but is not limited to, computer system 400, main memory 408, secondary memory 410, and removable storage units 418 and 422, as well as tangible articles of manufacture embodying any combination of the foregoing. Such control logic, when executed by one or more data processing devices (such as computer system 400), causes such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 4. In particular, embodiments can operate with software, hardware, and/or operating system implementations other than those described herein.

It is to be appreciated that the Detailed Description section, and not the Summary and Abstract sections, is intended to be used to interpret the claims. The Summary and Abstract sections can set forth one or more but not all exemplary embodiments as contemplated by the inventors, and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A method comprising:
receiving a request from a doctor to generate a medical diagnosis for a patient;
identifying, by one or more processors, one or more preconfigured templates corresponding to the medical diagnosis input by the doctor, wherein the identifying comprises matching one or more key words input by the doctor to a name of the one or more preconfigured templates;
receiving, from the doctor, a selection of at least one of the identified one or more preconfigured templates;
copying text of the selected at least one of the identified one or more preconfigured templates into a treatment plan for the patient; and
providing the treatment plan including at least a portion of the copied text of the selected at least one of the identified one or more preconfigured templates to the patient.

2. The method of claim 1, wherein the receiving input comprises:
receiving a selection of one or more check boxes, each check box corresponding to a different medical condition, wherein the one or more key words correspond to the selected medical condition.

3. The method of claim 2, wherein the receiving input comprises:
receiving a selection of one or more check boxes, each check box corresponding to a different medication, wherein the one or more key words correspond to the selected medication.

4. The method of claim 1, wherein the receiving input comprises:
identifying the one or more key words from text typed in by the doctor in a treatment plan box on a user interface.

5. The method of claim 1, further comprising:
receiving, via a user interface, one or more changes to the copied text, wherein the text of the selected at least one of the identified one or more preconfigured templates was copied into a treatment plan box of the user interface.

6. The method of claim 5, further comprising:
receiving, via a user interface, an indicate to update text of the selected at least one of the identified one or more preconfigured templates to reflect the one or more changes, wherein the one or more changes are available during a subsequent selection of the updated template.

7. The method of claim 1, wherein at least one of the one or more preconfigured templates was created by a different doctor.

8. The method of claim 1, wherein at least one of the one or more preconfigured templates was created by the doctor and shared with a different doctor.

9. A system comprising:
a memory; and
at least one processor coupled to the memory and configured to perform operations comprising:
receiving a request from a doctor to generate a medical diagnosis for a patient;
identifying, by one or more processors, one or more preconfigured templates corresponding to the medical diagnosis input by the doctor, wherein the identifying comprises matching one or more key words input by the doctor to a name of the one or more preconfigured templates;
receiving, from the doctor, a selection of at least one of the identified one or more preconfigured templates;
copying text of the selected at least one of the identified one or more preconfigured templates into a treatment plan for the patient; and
providing the treatment plan including at least a portion of the copied text of the selected at least one of the identified one or more preconfigured templates to the patient.

10. The system of claim 9, wherein the receiving input comprises:
receiving a selection of one or more check boxes, each check box corresponding to a different medical condition, wherein the one or more key words correspond to the selected medical condition.

11. The system of claim 10, wherein the receiving input comprises:
receiving a selection of one or more check boxes, each check box corresponding to a different medication, wherein the one or more key words correspond to the selected medication.

12. The system of claim 9, wherein the receiving input comprises:
identifying the one or more key words from text typed in by the doctor in a treatment plan box on a user interface.

13. The system of claim 9, the operations further comprising:
receiving, via a user interface, one or more changes to the copied text, wherein the text of the selected at least one of the identified one or more preconfigured templates was copied into a treatment plan box of the user interface.

14. The system of claim 13, the operations further comprising:
receiving, via a user interface, an indicate to update text of the selected at least one of the identified one or more preconfigured templates to reflect the one or more changes, wherein the one or more changes are available during a subsequent selection of the updated template.

15. The system of claim 9, wherein at least one of the one or more preconfigured templates was created by a different doctor.

16. The system of claim 9, wherein at least one of the one or more preconfigured templates was created by the doctor and shared with a different doctor.

17. A non-transitory computer-readable medium having instructions stored thereon that, when executed by at least one computing device, cause the at least one computing device to perform operations comprising:
receiving a request from a doctor to generate a medical diagnosis for a patient;
identifying, by one or more processors, one or more preconfigured templates corresponding to the medical diagnosis input by the doctor, wherein the identifying comprises matching one or more key words input by the doctor to a name of the one or more preconfigured templates;
receiving, from the doctor, a selection of at least one of the identified one or more preconfigured templates;
copying text of the selected at least one of the identified one or more preconfigured templates into a treatment plan for the patient; and
providing the treatment plan including at least a portion of the copied text of the selected at least one of the identified one or more preconfigured templates to the patient.

18. The non-transitory computer-readable medium of claim 17, wherein the receiving input comprises:
receiving a selection of one or more check boxes, each check box corresponding to a different medical condition, wherein the one or more key words correspond to the selected medical condition.

19. The non-transitory computer-readable medium of claim 18, wherein the receiving input comprises:
receiving a selection of one or more check boxes, each check box corresponding to a different medication, wherein the one or more key words correspond to the selected medication.

20. The non-transitory computer-readable medium of claim 17, wherein the receiving input comprises:
identifying the one or more key words from text typed in by the doctor in a treatment plan box on a user interface.
